(19) United States
(12) Patent Application Publication
(10) Pub. No.: US 2026/0171261 A1
(43) Pub. Date: Jun. 18, 2026

(54) MULTI-MODAL DATA AND FUSION MACHINE LEARNING FOR ROBOTIC MEDICAL SYSTEMS

(71) Applicant: Intuitive Surgical Operations, Inc., Sunnyvale, CA (US)

(72) Inventors: Ziheng Wang, Atlanta, GA (US); Samuel Max Berniker, San Francisco, CA (US); Shukai Chen, San Jose, CA (US); Sara Ivey Childs, Atlanta, GA (US); Rui Guo, Suwanee, GA (US); Anthony M. Jarc, Belmont, CA (US); Xi Liu, Peachtree Corners, GA (US); Conor Perreault, Atlanta, GA (US); Andrew Yee, Atlanta, GA (US)

(73) Assignee: Intuitive Surgical Operations, Inc., Sunnyvale, CA (US)

(21) Appl. No.: 19/419,863

(22) Filed: Dec. 15, 2025

Related U.S. Application Data
(60) Provisional application No. 63/734,667, filed on Dec. 16, 2024.

(51) Int. Cl. G16H 70/20; G06N 20/00

(52) U.S. Cl. CPC G16H 70/20; G06N 20/00

(57) ABSTRACT

Multi-modal data and ontology knowledge fusion machine learning for robotic medical systems is described. One or more processors can generate, using the training dataset and one or more teacher models, classifications of segments of the medical procedures in a first segment type. The one or more processors can map, using an ontology indicating a hierarchy of different segment types of medical procedures, the classifications of the segments in the first segment type to a second segment type. The one or more processors can train, using the mapping based on the classifications generated by the one or more teacher models, one or more student models with a machine learning technique. The one or more processors can execute, using data received from a robotic medical system for a medical procedure, the one or more student models to classify a segment of the medical procedure.

### CROSS-REFERENCE TO RELATED PATENT APPLICATION

This application claims the benefit of priority to U.S. Provisional Ser. No. 63/734,667, filed on Dec. 16, 2024, which is hereby incorporated by reference herein in its entirety for all purposes.

### BACKGROUND

A medical robotic system can include an instrument for performing a medical session or procedure. For example, the instrument can be used to perform surgery, therapy, or a medical evaluation. The medical robotic system can include an endoscope that captures a video of the medical procedure.

### SUMMARY

Technical solutions disclosed herein can include a computing system that trains procedure classification models specific to individual medical practitioners. For example, the technical solutions discussed herein can distill learning from teacher models trained on data of procedures of multiple medical practitioners to student models for individual medical practitioners. Furthermore, the technical solutions discussed herein can implement multi-modal data and fusion machine learning for robotic medical systems. The computing system can implement machine learning techniques using multi-modal data and hierarchical surgical ontology knowledge fusion to allow a model to accurately understand a surgical scene and recognize activities therein. With this accurate understanding of surgical scenes and activities, a robotic medical system can contribute to safer and more precise surgical procedures and interventions. At least one model can include a predefined mapping or matrix that maps between the different levels of the hierarchy. For example, the mapping can specify what types of tasks make up different steps, and what type of steps make up different phases. The model can generate classifications or predictions of a segment type at a first level (e.g., at the step level) and use the mapping to map the classification to a second level (e.g., the phase level). With the mapped classification, the computing system can use truth data to generate a loss to use in optimizing training of the model. The model system can be further trained with a fusion of multiple different data modalities (e.g., video data, robotic medical system event data, kinematics data, etc.). For example, the model system can be constructed to train and operate on data of different modalities such as endoscopic video data, system event data of a medical robotic system, robotic kinematic data, patient data, operating room data, etc. Using this fusion of data, the model system can improve the efficiency and accuracy of training and classification of segments of a medical procedure compared to models that consider a single data modality in isolation.

At least one aspect of the present disclosure is directed to a system. The system can include one or more processors, coupled with memory, to receive a training dataset related to medical procedures performed by one or more robotic medical systems. The one or more processors can generate, using the training dataset and one or more teacher models, classifications of segments of the medical procedures in a first segment type. The one or more processors map, using an ontology indicating a hierarchy of different segment types of medical procedures, the classifications of the segments in the first segment type to a second segment type. The one or more processors can train, using the mapping based on the classifications generated by the one or more teacher models, one or more student models with a machine learning technique. The one or more processors can execute, using data received from a robotic medical system for a medical procedure, the one or more student models to classify a segment of the medical procedure.

At least one aspect is directed to a system including one or more processors, coupled with memory, to receive a training dataset related to medical procedures performed by one or more robotic medical systems by medical practitioners. The one or more processors can generate, using the training dataset and one or more teacher models, classifications of segments of the medical procedures in a first segment type. The one or more processors can map, using an ontology indicating a hierarchy of different segment types of the medical procedures, the classifications of the segments in the first segment type to a second segment type. The one or more processors can train, using the mapping based on the classifications generated by the one or more teacher models and data associated with a single medical practitioner, one or more student models with a machine learning technique. The one or more processors can execute, using data received from a robotic medical system for a medical procedure, the one or more student models to generate a classification of a segment of the medical procedure performed by the single medical practitioner. The one or more processors can cause a graphical user interface to display the classification of the segment.

The ontology can indicate levels of different segment types. The levels can include at least two of a first level of actions and a mapping indicating steps that the actions map to, a second level of the steps and a mapping indicating phases that the steps map to, and a third level of the phases.

The one or more teacher models can store the ontology as at least one matrix.

The one or more processors can train the teacher model of the one or more teacher models to classify the segments of the medical procedure. The one or more processors can distill the training of the teacher model to a student model of the one or more student models. The one or more processors can execute, using the data received from the one or more robotic medical systems for the medical procedure, the student model to classify the segment of the medical procedure.

The one or more processors can train the teacher model using first data of the training dataset describing first medical procedures performed by medical practitioners via the one or more robotic medical systems. The one or more processors can distill the training of the teacher model using the first data to the student model. The one or more processors can train the student model using second data of the training dataset describing second medical procedures performed by a single medical practitioner via the one or more robotic medical systems. The one or more processors can execute, using the data received from the one or more robotic medical systems for a medical procedure performed by the single medical practitioner using the one or more robotic medical systems, the student model to classify the segment of the medical procedure.

The one or more processors can determine, using a first teacher model of the one or more models and the training dataset of a first data modality, first features. The one or more processors can classify, using the first teacher model, the segments using the first features. The one or more processors can determine, using a second teacher model of the one or more models and the training dataset of a second data modality, second features. The one or more processors can classify, using the second teacher model, the segments using the second features. The one or more processors can train the first teacher model and the second teacher model using one or more losses determined from the classification of the first teacher model and the classification of the second teacher model.

The first data modality or the second data modality can be a video data modality, a kinematics data modality, an event data modality.

The one or more processors can classify, using the first teacher model, the segments directly from the first features. The one or more processors can determine a first loss from the classified segments of the first teacher model and the training dataset. The one or more processors can train the first teacher model using the first loss.

The one or more processors can classify, using the second teacher model, the segments of a first level of the hierarchy from the second features. The one or more processors can map, according to the ontology indicating the hierarchy, the segments of the first level of the hierarchy to segments of a second level of the hierarchy, wherein the second level is higher than the first level in the hierarchy. The one or more processors can determine a second loss from the classify segments of the second teacher model and the training dataset. The one or more processors can train the second teacher model using the second loss.

The one or more processors can compare the first features with the second features. The one or more processors can train the first teacher model and the second teacher model to increase a dissimilarity between the first features and the second features.

The one or more processors can train the first teacher model and the second teacher model to maximize the dissimilarity between the first features and the second features.

The one or more processors can classify, using a model of the one or more models, segments of a first level of the hierarchy. The one or more processors can map, according to the ontology indicating the hierarchy, the classified segments of the first level of the hierarchy to segment types of a second level of the hierarchy, wherein the second level is higher than the first level in the hierarchy. The one or more processors can determine a loss using the classified segments mapped to the segment types of the second level of the hierarchy and the training dataset. The one or more processors can train the model using the loss.

The segments of the first level of the hierarchy can be steps of phases of the medical procedures. the segments of the second level of the hierarchy can be the phases of the medical procedures.

The model can be a teacher model and the one or more models include a student model. The one or more processors can distill the training of the teacher model to the student model.

The teacher model can include a first embedding model to generate first feature vectors from the training dataset. The first model can classify the segments of the first level from the first feature vectors. The student model can include a second embedding model to generate second feature vectors from the training dataset. The second model can classify the segments of the first level from the second feature vectors.

The one or more processors can determine a first loss for the teacher model based on the segments classified by the first model and the training dataset. The one or more processors can train the teacher model using the first loss. The one or more processors can determine a second loss for the student model based on the segments classified by the second model and the training dataset. The one or more processors can train the student model using the second loss.

The first loss can be a first cross-entropy loss. The second loss can be a second cross-entropy loss.

The one or more processors can compare the first feature vectors with the second feature vectors to generate a loss. The one or more processors can distill the training of the teacher model to the student model using the loss.

The one or more processors can generate distance measures between the first feature vectors and the second feature vectors. The one or more processors can update at least one parameter of the second embedding model of the student model to decrease the distance measures.

The one or more processors can update the at least one parameter of the second embedding model to minimize the distance measures.

At least one aspect of the present disclosure is directed to a method. The method can include receiving, by one or more processors, coupled with memory, a training dataset describing medical procedures performed by one or more robotic medical systems. The method can include training, by the one or more processors, using the training dataset and an ontology indicating a hierarchy of different segment types of medical procedures, a teacher model to classify segments of the medical procedures. The method can include distilling, by the one or more processors, the training of the teacher model to a student model trained to classify the segments of the medical procedures. The method can include executing, using data received from a one or more robotic medical system for a medical procedure, the student model to classify a segment of the medical procedure.

At least one aspect of the present disclosure is directed to a method. The method can include receiving, by one or more processors, coupled with memory, a training dataset describing medical procedures performed by one or more robotic medical systems by medical practitioners. The method can include training, by the one or more processors, using the training dataset and an ontology indicating a hierarchy of different segment types of the medical procedures, a teacher model to classify segments of the medical procedures. The method can include distilling, by the one or more processors, the training of the teacher model to a student model trained to classify the segments of the medical procedures for a single medical practitioner. The method can include executing, using data received from a one or more robotic medical system for a medical procedure, the student model to generate a classification of a segment of the medical procedure for the single medical practitioner. The method can include causing, by the one or more processors, a graphical user interface to display the classification of the segment of the medical procedure.

The method can include determining, by the one or more processors, using a first teacher model and the training dataset of a first data modality, first features. The method can include classifying, by the one or more processors, using the first teacher model, the segments using the first features. The method can include determining, by the one or more processors, using a second teacher model and the training dataset of a second data modality, second features. The method can include classifying, by the one or more processors, using the second teacher model, the segments using the second features. The method can include training, by the one or more processors, the first teacher model and the second teacher model using one or more losses determined from the classification of the first teacher model and the classification of the second teacher model.

The first data modality or the second data modality are a video data modality, a kinematics data modality, an event data modality.

The method can include classifying, by the one or more processors, using the first teacher model, the segments directly from the first features. The method can include determining, by the one or more processors, a first loss from the classified segments of the first teacher model and the training dataset. The method can include training, by the one or more processors, the first teacher model using the first loss. The method can include classifying, by the one or more processors, using the second teacher model, the segments of a first level of the hierarchy from the second features. The method can include mapping, by the one or more processors, according to the ontology indicating the hierarchy, the classified segments of the first level of the hierarchy to segment types of a second level of the hierarchy, wherein the second level is higher than the first level in the hierarchy. The method can include determining, by the one or more processors, a second loss from the classified segments mapped to the segment types of the second level and the training dataset. The method can include training, by the one or more processors, the second teacher model using the second loss.

At least one aspect of the present disclosure is directed to one or more storage media storing instructions thereon, that, when executed by one or more processors, cause the one or more processors can receive a training dataset including data of a first data modality and data of a second data modality, the training dataset describing medical procedures performed by one or more robotic medical systems. The one or more processors can train one or more models with a machine learning technique to classify segments of the medical procedures using the data of the first data modality and the data of the second data modality. The one or more processors can execute, using inference data of the first data modality and inference data of the second data modality received from a robotic medical system for a medical procedure, the one or more models to classify a segment of the medical procedure.

At least one aspect of the present disclosure is directed to a non-transitory computer-readable medium storing processor-executable instructions that, when executed by one or more processors, cause the one or more processors to receive a training dataset related to medical procedures performed by one or more robotic medical systems by medical practitioners. The instructions can cause the one or more processors to generate, using the training dataset and one or more teacher models, classifications of segments of the medical procedures in a first segment type. The instructions can cause the one or more processors to map, using an ontology indicating a hierarchy of different segment types of the medical procedures, the classifications of the segments in the first segment type to a second segment type. The instructions can cause the one or more processors to train, using the mapping based on the classifications generated by the one or more teacher models and data associated with a single medical practitioner, one or more student models with a machine learning technique. The instructions can cause the one or more processors to execute, using data received from a robotic medical system for a medical procedure, the one or more student models to generate a classification of a segment of the medical procedure performed by the single medical practitioner. The instructions can cause the one or more processors to cause a graphical user interface to display the classification of the segment.

The ontology can indicate levels of different segment types, wherein the levels include at least two of a first level of actions and a mapping indicating steps that the actions map to, a second level of the steps and a mapping indicating phases that the steps map to, and a third level of the phases.

The one or more processors can classify, using a model of the one or more models, segments of a first level of a hierarchy. The one or more processors can map, according to an ontology indicating a hierarchy of different segment types of medical procedures, the classified segments of the first level of the hierarchy to segment types of a second level of the hierarchy, wherein the second level is higher than the first level in the hierarchy. The one or more processors can determine a loss using the segments mapped to the second level of the hierarchy and the training dataset. The one or more processors can train the model using the loss.

The segments of the first level of the hierarchy can be steps of phases of the medical procedure. The segments of the second level of the hierarchy are the phases of the medical procedure.

These and other aspects and implementations are discussed in detail below. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification. The foregoing information and the following detailed description and drawings include illustrative examples and should not be considered as limiting.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 depicts an example computing system to train a machine learning model using ontology knowledge.

FIG. 2 depicts an example computing system to train a machine learning model using ontology knowledge and data of multiple modalities.

FIG. 3 depicts an ontology of different segment types of a medical procedure organized in a hierarchy.

FIG. 4 depicts an example method of training a machine learning model using ontology knowledge.

FIG. 5 depicts an example computing architecture of a computing system.

### DETAILED DESCRIPTION

Following below are more detailed descriptions of various concepts related to, and implementations of, methods, apparatuses, and systems for multi-modal data and fusion machine learning for robotic medical systems. The various concepts introduced above and discussed in greater detail below may be implemented in any of numerous ways.

This disclosure is generally directed to surgical scene understanding using machine learning models. A medical or surgical procedure can be performed by a robotic medical system, which can include an endoscope that records a video of the medical procedure. Providing the robotic medical system with labels or indications generated by a machine learning model that classify different segments of the medical procedure video and provide an understanding of surgical scenes and activities and improve the performance of the robotic medical system, and improve outcomes of the robotic medical system.

The machine learning models can be designed and trained to detect or classify segments of a medical procedure video. For example, for a segment of the video, a model can be trained to detect a phase of the medical procedure, a step of the medical procedure, an action of the medical procedure, or gesture by a robotic arm in the video. However, the model may not be trained take into account the hierarchical nature of surgical ontologies, i.e., different phases are made up of different steps, different steps are made up of different actions, different actions are made up of different gestures. Without this ontological context, the model may not be able to accurately classify a segment of the video. In particular, the model may not be able to discern fine-grained details of the video, such as granular and atomic actions and gestures. Furthermore, the models may rely on a single data modality (e.g., video data, kinematics data, event etc.) to attempt to classify a segment of the video. A model relying on a single data modality may be limited in its ability to accurately capture the complexity of surgical procedures.

To solve these, and other technical problems, technical solutions of this disclosure can include a computing system that implements multi-modal data and ontology knowledge fusion machine learning for robotic medical systems. Technical solutions disclosed herein can include a computing system that trains procedure classification models specific to individual medical practitioners. For example, the technical solutions discussed herein can distill learning from teacher models trained on data of procedures of multiple medical practitioners to student models for individual medical practitioners. The computing system can implement machine learning techniques using multi-modal data and hierarchical surgical ontology knowledge fusion to allow a model to accurately understand a surgical scene and recognize activities therein. With this accurate understanding of surgical scenes and activities, a robotic medical system can contribute to safer and more precise surgical procedures and interventions.

The computing system can incorporate the hierarchy of segment types of medical procedures into the training of the models. For example, the computing system can train and execute a machine learning model system using an ontological definition of a hierarchy of segment types (e.g., phases, steps, actions, gestures, etc.) to classify segments of a medical procedure performed by a medical robotic system. The multiple levels of surgical ontologies can provide supplementary descriptions of the same subjects, for example, a particular medical phase can be formed from a sequence of predefined steps. A supplementary description of one of the steps can be the phase that the one step corresponds to. Therefore, correlating the two descriptions together during a learning phase can result in more informative model training, thereby improving the performance and accuracy of the model.

At least one model can include a predefined mapping or matrix that maps between the different levels of the hierarchy. For example, the mapping can specify what types of tasks make up different steps, and what type of steps make up different phases. The model can generate classifications or predictions of a segment type at a first level (e.g., at the step level) and use the mapping to map the classification to a second level (e.g., the phase level). With the mapped classification, the computing system can use truth data to generate a loss to use in optimizing training of the model. By incorporating an understanding of the ontological hierarchical relationships between segment types in the training of the model, the model can result in increased accuracy and efficiency compared to models that only consider single levels of the ontology in isolation.

The model system can be further trained with a fusion of multiple different data modalities (e.g., video data, robotic medical system event data, kinematics data, etc.). For example, the model system can be constructed to train and operate on data of different modalities such as endoscopic video data, system event data of a medical robotic system, robotic kinematic data, patient data, operating room data, etc. Using this fusion of data, the model system can improve the efficiency and accuracy of training and classification of segments of a medical procedure compared to models that consider a single data modality in isolation.

Furthermore, the computing system can distill learning of a teacher model to a student model. The teacher model can continuously train as new training data is collected, and periodically distill learning of the teacher model to the student model. The student model can be deployed to execute to classify segments of a medical procedure, and can be tuned using a tailored dataset (e.g., a data set of data for one specific medical practitioner, one specific operating room, one specific medical robotic system, etc.). This teach-student learning framework can enhance the model system's learning process and overall performance. For example, the framework can reduce the need for fine-grained ontology annotations in a training dataset because the initial information can be learned from knowledge distillation gained from initial pretraining stage. Furthermore, using the mapping from a lower level of segment type (such as a gesture or action) that does not appear frequently in a training dataset to a higher level of segment type (such as phase) which appears more frequently in the training dataset, the system can still train the model to predict the lower level segment by computing a loss with the mapped higher level segment type for training, even though the training dataset has no or a small amount of data labeled at the low level segment. Furthermore, an ontology dependency loss included in the framework can increase the agreement between ontologies in different levels of the hierarchy. The framework can increase model performance through total information increase (additional data streams) and collaborative learning.

The techniques of the present disclosure can leverage multi-modal data fusion and a hierarchical surgical ontology to deliver significant clinical and operational benefits. By integrating video, kinematic, and event data with ontology-driven models, the present techniques can achieve higher accuracy in surgical scene understanding and robust classification of gestures, actions, steps, and phases. This can enable real-time (or near real-time) recognition and prediction of surgical segments intraoperatively, thus improving precision in robotic and manual procedures. Furthermore, ontology-based mapping can allow inference of higher-level surgical context even from sparse data, supporting structured pre-operative planning and intra-operative decision-making. These capabilities can enhance clinical outcomes, reduce variability, and provide surgeons with actionable insights during complex procedures.

Furthermore, the present system can transform surgeon experience and skill development through adaptive learning frameworks. A teacher-student architecture can enable scalable training and personalized model fine-tuning based on individual styles and skill levels. Knowledge distillation from large datasets can accelerate skill enhancement without requiring extensive personal data, while dynamic loss scheduling can reinforce conceptual learning early in training. Real-time intra-operative guidance, automated segment classification, and intuitive graphical can reduce cognitive load and streamline workflows. Furthermore, continuous model evolution can ensure adaptability to new techniques, supporting long-term performance, surgeon training, and benchmarking, ultimately fostering a safer, more efficient surgical environment.

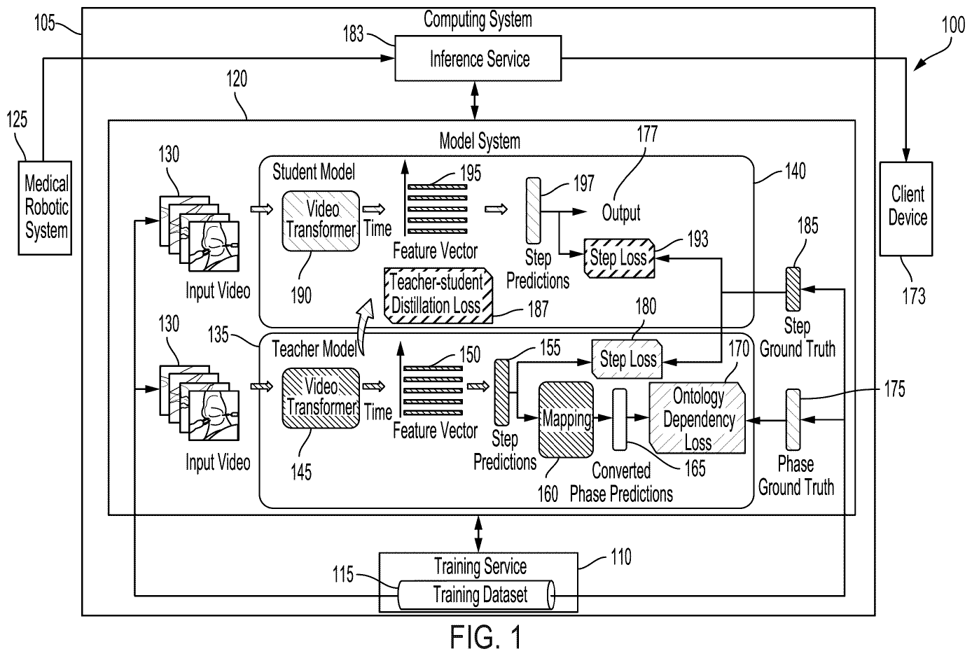

Referring now to FIG. 1, among others, an example system 100 including at least one computing system 105 to train a machine learning model 120 using ontology knowledge is shown. The computing system 105 can be a data processing system, a computing system, a computer system, a computer, a desktop computer, a laptop computer, a tablet, a control system, a console system, an embedded system, a cloud computing system, a server system, or any other type of computing system. The computing system 105 can be an on-premises system or an off-premises system. The computing system 105 can be a hybrid system, where some components of the computing system 105 are located on-premises, and some components of the computing system 105 are located off-premises.

The system 100 can include at least one medical robotic system 125. The medical robotic system 125 can be a robotic system, apparatus, or assembly including at least one instrument. The instrument can be or include a tip or end. The tip or end can be installed with or to the instrument. The tip can be removable or a permanent component of the instrument or the medical robotic system 125. For example, the tip can be a scalpel, a scissors, a monopolar curved scissors (MCS), a cautery hook tip, a cautery spatula tip, a needle driver, a forceps, a round tooth retractor, a drill, or a clip applier. The instrument can be or include a robotic arm, a robotic appendage, a robotic snake, or any other motor controlled member that can be articulated by the medical robotic system 125. The instrument can include at least one actuator, such as a motor, servo, or other actuator device. The instrument can be manipulated by motors, servos, actuators, or other devices to perform a medical procedure. The medical robotic system 125 can perform a medical session or medical procedure. For example, the medical robotic system 125 can articulate the instrument to perform surgery, therapy, or a medical evaluation with the instrument. The medical procedure can be performed on a subject, e.g., a human, an adult, a child, or an animal. A medical practitioner, such as a surgeon, technician, nurse, or other operator can provide input via a user device or input apparatus (e.g., joystick, buttons, touchpad, keyboard, steering apparatus, etc.) to manipulate the instrument to perform a medical procedure. The medical robotic system 125 can include an endoscope, in some implementations. The endoscope can be an instrument that is manipulated by the medical practitioner and controlled via a motor, servo, or other input device.

The computing system 105 can include at least one training service 110. The training service 110 can be or include a software component (e.g., a program, a module, an object, etc.) or a hardware component (e.g., a hardware server, a computing device, a graphics processing unit (GPU), a neural processing unit (NPU), etc.). The training service 110 can perform at least one machine learning or training technique to train at least one model system 120. For example, the training service 110 can perform backpropagation and to minimize or maximize losses to train the model system 120. For example, the training service 110 can execute a machine learning algorithm, such as gradient descent of losses or stochastic gradient descent of the losses with respect to parameters of the model system 120. The machine learning algorithm can implement second order gradient descent, newton method, conjugate gradient, quasi-newton method, or Levenberg-Marquardt algorithm to train the model system 120.

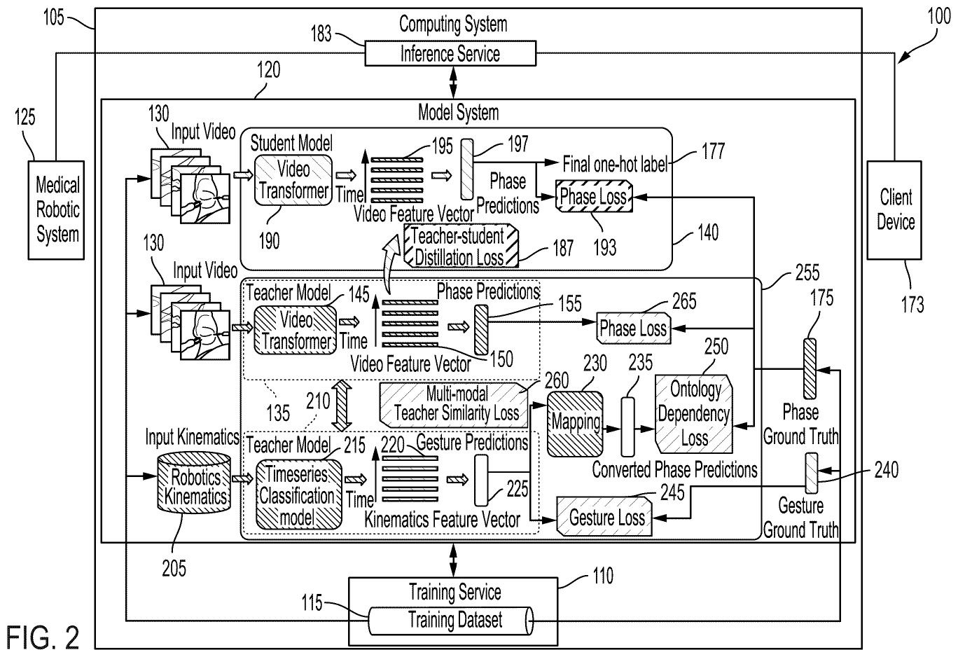

The computing system 105, or the training service 110, can receive the training dataset 115. The training service 110 can include or store the training dataset 115. The data of the training dataset 115 can be related to at least one medical procedures performed by at least one robotic medical systems 125. For example, the training dataset 115 can include data describing different medical procedures performed by at least one medical robotic system 125 or a group of different medical robotic systems 125. For example, the training dataset 115 can include data collected by medical robotic systems 125 while performing a medical procedure. The training dataset 115 can include data of a variety of data modalities, for example, video data 130, event data, kinematics data 205 (shown in FIG. 2), operating room (OR) data, etc.

The training service 110 can use the training dataset 115 including the various data modalities to train a model system 120 that operates on a single data modality or multiple data modalities. In FIG. 1, the computing system 105 can provide single-modality learning with knowledge distillation of surgical ontologies. In FIG. 1. The model system 120 can provide a modeling process for surgical step recognition using a hierarchical surgical ontology. The single modality can be endoscopic video, and the model system 120 can integrate information of two different surgical ontology levels, universal phases and procedure specific steps, for learning.

For example, the video data 130 of the training dataset 115 can be or include high-resolution (e.g., 720p, 1080p, 2k, 4k, etc.) endoscopic video recordings that can capture visual nuances of a surgical procedure. The video data 130 can include a series of frames, such as surgical or medical procedure images. The frames can be represented as continuous-valued matrices. The video data 130 can enable the model system 120 to understand macro-level aspects of a surgical procedure. The training dataset 115 can include event data generated by the medical robotic systems 125. The event data can be events that represent actions or occurrences in the medical robotic system 125. The events can include data values, descriptions of the events, time-steps when the events occurred, etc. The events can represent actions performed by an operator when operating the medical robotic system 125. For example, the event count be a stapler being used, an operator pressing a clutch or brake of the medical robotic system 125, an operator causing a forceps of the medical robotic system 125 to close, etc. The events can indicate conditions measured by the medical robotic system 125. For example, the event can indicate an energy usage level for the medical robotic system 125 to coagulate tissue, a linear distance an instrument has traveled, a rotational distance an instrument has rotated, etc. The event data modality can enrich the model system 120 to understand dynamics of the surgical field.

The kinematics data 205 can be or include information, data, data frames, or values collected by or from the robotic medical systems 125 when performing the medical procedure. The kinematics data 205 can be time correlated data, e.g., data with timestamps such as a timeseries. The kinematics data 205 can be time correlated with the frames of the video 130. The kinematics data 205 can be or include force, torque, acceleration, linear movement, angular movement, positions, or velocity data of collective or individual joints, links, arms, appendages, manipulators, patient-side robotic arms or instruments, or surgeon side robotic arms or instruments of the robotic medical system 125. For example, the kinematics data 205 can identify a series of positions in three dimensional space of an active robotic instrument of the robotic medical system 125. The kinematics data 205 can be captured or recorded from at least one sensor associated with the scene of the medical procedure. For example, the robotic medical systems 125 can include sensors such as encoders, tachometers, current sensors, power meters, or force sensors. The kinematic data 205 can provide fine-grained details about the motions and interactions during surgery, contributing to a more detailed analysis of surgical tasks or actions by the model system 120.

The training dataset 115 can include OR data. The OR data can be data collected or generated by at least one system that controls or monitors an operating room. The OR data can indicate procedure schedules, surgeon schedules, patient information, video surveillance of the operating room, etc. The OR data can indicate the setup or design of the operating room, e.g., indicate what surgical tools or equipment are being used in the operating room. The OR data can indicate surgical histories of patients. The OR data can indicate patient comorbidities, patient health, medications taken by the patient, etc.

The model system 120 can have a teacher-student model topology. The model system 120 can include at least one student model 140 and at least one teacher model 135. In FIG. 1, the model system 120 can operate on a single-modality, e.g., video data. In FIG. 1, the model system 120 can be configured to detect surgical steps of a medical procedure using a hierarchical surgical ontology. However, the model system 120 can be used to detect any segment type of a medical procedure video, e.g., detect procedure, phase, task, action, gesture, etc. The model system 120 can include at least one student model 140. The model system 120 can include at least one teacher model 135. The student model 140 and the teacher model 135 can learn separately. The training service 110 can first pretrain the teacher model 135 and the transfer knowledge from the trained teacher model 135 to the student model 140. In some embodiments, the training service 110 can simultaneously pretrain the teacher model 135 and/or student model 140 and distill knowledge from the teacher model 135 to the student model 140. Once pretrained, in addition to training a single output teacher 135 as shown in FIG. 1, a multi-output student 140 can be built to output predictions of phase and step all together. For example, the student model 140 can include multiple outputs 177, e.g., can predict a gesture, step, action, phase, or procedure type.

The teacher model 135 can include at least one feature extraction model or embedding model, such as a video transformer 145, that generates at least one feature vector 150 from at least one input frame of a video 130. Depending on the data modality which the extraction model 145 determines features, the model 145 can be a variety of forms, for example, for video classification, the model 145 can be a vision transformer based architecture, such as Timesformer, a convolutional neural network (CNN), an LSTM, etc. The transformer 145 can be a video transformer 145 that includes a CNN, an attention mechanism, and a temporal transformer. The video transformer 145 can receive at least one frame of a longer video 130 of the training dataset 115. For example, during a training phase of the teacher model 135, the training service 110 can feed at least one frame into the teacher model 135. The video transformer 145 can receive a window of frames of the video 130, e.g., a 15-17 second window, a 10-20 second window, a window less than 10 seconds, a window more than 20 seconds. In some embodiments, the video transformer 145 can receive a single frame at a time, or may receive an entire frame set of a video 130.

The video transformer 145 can output feature vectors 150 based on the frames of the video 130. The feature vectors 150 can each be generated for one input segment or window of the video 130. In this regard, the teacher model 135 can store the feature vectors 150 in an order corresponding to timestamps or corresponding to the windows for which the feature vectors 150 were generated. Each feature vector 150 can be a compressed or lower dimensional representation of information in the window of frames.

The computing system 105 can generate, using the training dataset 115 and the teacher model 135, classifications of segments of the medical procedures in a first segment type. The teacher model 135 can include at least one model to generate predictions 155. The teacher model 135 can include a support vector machine, a decision tree, a neural network, a convolutional neural network, a recurrent neural network, etc. to generate a prediction or classification 155 using the feature vectors 150. For example, the model can be a classification model that generates a classification of the feature vectors 150 into a first segment type. The different segment types can be defined by an ontology, and can be or include procedure type, phase, step, action, or gesture. The training service 110 can pretrain the teacher model 135. In some embodiments, the training service 110 can pretrain the teacher model 135 on a large dataset of surgical videos across multiple procedure types. The teacher model 135 an include a loss formed from two components, a cross entropy loss 180 based on direct predictions and ground truth, and an ontology dependency loss based on converted phase predictions 165 via step-phase ontology mapping and phase ground truth 175.

The computing system can train and deploy a model system 120 using an ontology of segment types and the hierarchical relationships between the segment types. The ontology can be included in at least one model of the model system 120 and can be used to train the model system 120. The ontology can be incorporated into the model 120 as a mapping 160 (e.g., a matrix) between hierarchy levels. The hierarchy levels can include (from lowest level to highest level), gestures, actions, steps, phases, and procedure type. The ontology can be predefined, and indicate that specific gestures correspond to specific actions, and that specific actions correspond to specific tasks, and that specific tasks correspond to specific phases, and that specific phases correspond to a specific procedure type.

The prediction 155 made by the teacher model 135 using the feature vectors 150 can be at any level of the hierarchy. The prediction 155 that the teacher model 135 makes can be at a level lower than the top level. The model system 120 can predict segments at a low level, and use the ontology mapping 160 to map the predicted segment to a higher level, e.g., the model 135 can predict a task and use the mapping 160 to identify a corresponding phase for the task. For example, in FIG. 1, the teacher model 135 can generate a prediction of a step 155 using the feature vectors 150.

The computing system 105 can map, using an ontology indicating a hierarchy of different segment types of medical procedures, the classifications 155 of the segments in the first segment type to a second segment type. The ontology can define available segment types and the hierarchy of the segment types, e.g., what level each segment types fall within. The ontology can define various gestures that can be part of medical procedures, various actions that can be part of medical procedures, various phases that can be part of medical procedures, or various overall types of the medical procedures.

The teacher model 135 can store the ontology in the mapping 160. The teacher model 135 can include a mapping 160 that includes or is based on the ontology. The mapping 160 can define a hierarchy or a translation between the segment types. For example, the mapping 160 can indicate that a particular step of a variety of different steps of the ontology are part of or form a particular phase of a variety of different phases of the ontology. The mapping 160 can be a matrix, e.g., a two dimensional (2D) matrix. For example, the mapping 160 can translate predictions 155 from one level of the hierarchy to another level of the hierarchy, and therefore, the matrix can be a two dimensional matrix. The mapping 160 can map or convert between levels of the hierarchy, e.g., from a lower level to a higher level. In FIG. 1, the 2D matrix can map from a step prediction to a phase prediction. In FIG. 1, the step prediction 155 can be mapped or converted to phase predictions 165. If the mapping 160 translates predictions from one level, to a second level, and then to a third level, the mapping 160 can be a three dimensional matrix or there can be two separate 2D matrixes (e.g., one 2D matrix for the first translation, and one 2D matrix for the second translation).

The teacher model 135 can train, using the mapping based on the classifications generated by the one or more teacher models 135, one or more student models with a machine learning technique. Training the student model 140 can include training the teacher model 135, and then distilling the training to the student model 140. For example, the training service 110 can train the teacher model 135 to classify segments of a medical procedure. The training service 110 can train the teacher model 135 using the converted predictions 165. The model system 120 can use truth data of the training dataset 115 to generate a loss 170 using the mapped segment, and train the model using the resulting loss 170. For example, the training service 110 can determine an ontology dependency loss 170. The loss 170 can be mean absolute error (MAE), mean squared error (MSE), cross-entropy loss, etc. The training service 110 can determine the loss 170 based on the converted predictions 165 and ground truth 175. For example, the training service 110 can compare the converted predictions 165 with the ground truth 175 to compute the loss. The ground truth 175 can be a predetermined classification or label of the segment. The ground truth 175 can be a classification of the segment in the same level of the hierarchy as the converted prediction 165. For example, the ground truth 175 can indicate the actual phase for the segment that the teacher model 135 converted the step predictions 155 to. For example, the converted prediction 165 can be a predicted phase for the segment, while the ground truth 175 can indicate the actual phase for the segment.

The training service 110 can determine a loss 180 using the predictions 155 and ground truth 185. The loss 185 can be mean absolute error (MAE), mean squared error (MSE), cross-entropy loss, etc. The training service 110 can compare the predictions 155 with the ground truth 185 to compute the loss 180. The ground truth 185 can be a predetermined classification of the segment. The ground truth 185 can be a classification of the segment in the same level of the hierarchy as the prediction 155. For example, the ground truth 185 can indicate the actual step for the segment that the teacher model 135 directly predicted. For example, the prediction 155 can be a predicted step for the segment, while the ground truth 185 can indicate the actual step for the segment.

The training service 110 can train the teacher model 135 using at least one loss. For example, the training service 110 can train the teacher model 135 using the loss 180 and the loss 170. For example, the training service 110 can execute at least one machine learning technique to train the teacher model 135 using the loss 180 and the loss 170. For example, the training service 110 can minimize the loss 180 and the loss 170. The training service 110 can execute a machine learning algorithm, such as gradient descent of the loss 180 and the loss 170 or stochastic gradient descent of the loss 180 and the loss 170 with respect to parameters of the teacher model 135. The machine learning algorithm can implement second order gradient descent, newton method, conjugate gradient, quasi-newton method, or Levenberg-Marquardt algorithm to train the teacher model 135. The machine learning algorithm can adjust, change, tune, or train the parameters or weights of the video transformer 145 or the model of the teacher model 135 used to generate the predictions 155.

The student model 140 can include at least one embedding model, such as a video transformer 190, that generates at least one feature vector 195 from at least one input frame of a video 130. The transformer 190 can be a video transformer 190 that includes a convolutional neural network (CNN), an attention mechanism, and a temporal transformer. The video transformer 190 can receive at least one frame of a longer video 130 of the training dataset 115. For example, during a training phase of the student model 140, the training service 110 can feed at least one frame into the student model 140. The video transformer 190 can receive a window of frames of the video 130, e.g., a 15-17 second window, a 10-20 second window, a window less than 10 seconds, a window more than 20 seconds. In some embodiments, the video transformer 190 can receive a single frame at a time, or may receive an entire frame set of a video 130.

The video transformer 190 can output feature vectors 195 based on the frames of the video 130. The feature vectors 195 can each be generated for one input segment or window of the video 130. In this regard, the student model 140 can store the feature vectors 195 in an order corresponding to timestamps or corresponding to the windows for which the feature vectors 195 were generated. Each feature vector 195 can be a compressed or lower dimensional representation of information in the window of frames.

The student model 140 can include at least one model to generate predictions 197. For example, the model can be a classification model that generates a classification of the feature vectors 195 into a first segment type. The student model 140 can include a support vector machine, a decision tree, a neural network, a convolutional neural network, a recurrent neural network, etc. to classify or predict the step 197 using the feature vectors 195. The different segment types can be defined by an ontology, and can be or include procedure type, phase, step, action, or gesture. Each procedure type can be one level of a hierarchy defined by an ontology. For example, the ontology can define an order of hierarchical levels, e.g., procedure type can be at a top level, phase can be at a lower level, step can be a yet a lower level, action can be at yet a lower level, and gesture can be at yet a lower level. The prediction 197 made by the student model 140 generates using the feature vectors 195 can be at any level of the hierarchy. The prediction 197 that the student model 140 makes can be at a level lower than the top level. For example, in FIG. 1, the student model 140 can generate a prediction 197 of a step using the feature vectors 195.

The training service 110 can train the student model 140 using the predictions 197. For example, the training service 110 can determine a loss 193. The loss 193 can be mean absolute error (MAE), mean squared error (MSE), cross-entropy loss, etc. The training service 110 can determine the loss 193 based on the predictions 197 and ground truth 185. For example, the training service 110 can compare the predictions 197 with the ground truth 185 to compute the loss. The ground truth 185 can be a predetermined classification of the segment. The ground truth 185 can be a classification of the segment in the same level of the hierarchy as the prediction 197. For example, the ground truth 185 can indicate the actual step for the segment that the student model 140 predicted. For example, the prediction 197 can be a predicted step for the segment, while the ground truth 185 can indicate the actual step for the segment.

The training service 110 can train the student model 140 using at least one loss. For example, the training service 110 can train the student model 140 using the loss 193. For example, the training service 110 can execute at least one machine learning technique to train the student model 140 using the loss 187. For example, the training service 110 can minimize the loss 193. The training service 110 can execute a machine learning algorithm, such as gradient descent of the loss 193 or stochastic gradient descent of the loss 193 with respect to parameters of the student model 140. The machine learning algorithm can implement second order gradient descent, newton method, conjugate gradient, quasi-newton method, or Levenberg-Marquardt algorithm to train the student model 140. The machine learning algorithm can adjust, change, tune, or train the parameters or weights of the video transformer 190 or the model of the student model 140 used to generate the predictions 197 to reduce or minimize the loss 187. By adjusting the parameters of the video transformer 190 to minimize the loss 187, the feature vectors 195 generated by the video transformer 190 can become more similar to the feature vectors 150 generated by the video transformer 145 for the same segment of the video 130.

The training service 110 can distill the training of the teacher model 135 to a student model 140. The training service 110 can determine a teacher-student distillation loss 187, and distill the training of the teacher model 135 to the student model 140. The training service 110 can determine the loss 187 using the feature vectors 150 and the feature vectors 195. The training service 110 can compare the feature vectors 150 with the feature vectors 195 to determine or generate a value for the loss 187. The loss 187 can be a distance measure between at least one feature vector 150 and at least one corresponding feature vector 195. The loss 187 can be Euclidean distance, Manhattan Distance, Cosine Similarity, Kullback-Leibler (KL) divergence, etc. Each value of the loss 187 can be determined from at least one feature vector 150 and at least one feature vector 195 generated for the same segment of the video 130. For example, for a given frame or set of frames of the video 130, the video transformer 145 can generate at least one feature vector 150 and the video transformer 190 can generate at least one feature vector 195. These feature vectors 150 and 195 can be compared against each other to determine the loss 187. The teacher-student distillation loss 187 can encourage the student model 140 to mimic the teacher model 135. For example, the loss 187 can encourage similarity between feature vectors, attention maps, and/or decision layers of classification probabilities. The distillation can be a separate training stage from training the teacher model 135, or can be combined with the teacher training or pretraining. For example, one or more teacher models 135 and one or more student models 140 can be trained separately by first pretraining the one or more teacher models 135, and then distilling knowledge to the one or more student models 140 once the pretraining is completed. Alternatively, the training service 110 can combine pretraining and distilling together into a single learning stage. In some embodiments, the loss 187 and the loss 193 can be used together to train the student model 140.

In some implementations, the training service 110 can train the teacher model 135 and the student model 140 using different training datasets 115. For example, the different training datasets 115 can allow for a surgeon specific student model 140 to be trained to classify segments specific to the surgeon. For example, the teacher model 135 can be trained on a first training dataset 115 for a variety of different surgeons, while the student model 140 can be tuned with a second training dataset 115 specific to one surgeon. The student model 140 can be tuned or optimized for other characteristics or attributes besides surgeon identity, such as site or facility, geography, surgeon skill level, medical procedure complexity, etc. In some embodiments, the client device 173 can display a graphical user interface, within which a user can provide input to select one particular student model 140 to use, or provide input to identify the characteristic for the student model 140 to be tuned for. By using a student model 140 tuned for a specific surgeon, for example, this technical solution can improve the operation of a robotic medical system, such as by improving the accuracy, efficiency, reliability or safety of the operation, without using excessive computing resources that may be utilized by a larger machine learning model.

For example, the training service 110 can train the teacher model 135 using first data of a first training dataset 115 and train or fine-tune the student model 140 using second data of a second training dataset. The knowledge learned by training the teacher model 135 using the first training dataset 115 can be distilled or transferred to the student model 140. In some embodiments, the first training dataset 115 can be data describing medical procedures performed by multiple different practitioners with one or multiple different medical robotic systems 125. However, the second training dataset 115 can be data describing medical procedures performed by one single medical practitioner. In this regard, the teacher model 135 can distill knowledge for a large group of medical practitioners to the student model 140, but the student model 140 can be tuned to make predictions specific to one individual medical practitioner. The student model 140 can be deployed to run or execute for the one specific medical practitioner.

In some embodiments, the first training dataset 115 and the second training dataset 115 are different sizes. For example, the first training dataset 115 used to train the teacher model 135 can be larger (e.g., include data of more medical procedures, include more data samples, etc.) than the second training dataset 115 used to train the student model 140. In some embodiments, the second training dataset 115 can be half, a quarter, or a third the size of the first training dataset 115. In this regard, the larger size of the first training dataset 115 can be used to accurately train the teacher model 135, and the smaller second training dataset 115 can tune the student model 140. The teacher model 135 can be trained on a larger training dataset 115, while the student model 140 can be fine-tuned on a smaller dataset of labeled surgical steps. The student model 140 can be fine-tuned on a specific task in a certain type of procedure, such as dissection of gallbladder off liver bed in robotic cholecystectomy. This can include using a dynamic loss function to focus on different aspects of the task at different stages of fine-tuning training. For example, the training service 110 can schedule a dynamic loss that depends on (or changes based on) training epoch. The dynamic loss can assign higher weights to the phase ontology dependency loss at the early stage of training to encourage model to focus more on surgical ontology information. The selection of teacher models 135 and importance factors can be pre-determined based on certain prior knowledge of data itself, or dynamically adjusted as part of training process based on their collaborative goal in order to produce a suitable shared knowledge that the student can effectively mimic.

The computing system 105 can include at least one inference service 183. The inference service 183 can deploy the student model 140 responsive to the student model 140 being trained. The inference service 183 can execute the student model 140 to generate inferences or predictions. The inference service 183 can receive data from the medical robotic system 125 for a particular medical procedure performed by the medical robotic system 125. The data received from the medical robotic system 125 can be endoscope video data, event data, kinematics data, OR data, etc. The inference service 183 can execute the student model 140 using the received data to generate a prediction 197. The prediction 197 can be provided as an output 177 of the student model 140. The output 177 can be a tag, label, or one-hot encoding or label of a particular segment of the video 130. The model system 120 can provide the output 177 to the medical robotic system 125 or a client device 173. For example, the classifications of the student model 140 at inference (or at training) can be displayed on a graphical user interface by the client device 173. Alternatively, the graphical user interface can be displayed on the medical robotic system 125.

In some embodiments, the training service 110 can continuously or periodically train and retrain the teacher model 135 over time. For example, the training service 110 can collect training datasets 115 from various medical robotic systems 125. A user can provide, via the client device 173, label input or truth data identifying the procedure type, phases, steps, actions, or gestures of various segments of the medical procedures. Responsive to a predefined amount of new training data being received (e.g., data of a predefined number of procedures or a predefined number of data samples being received) or a predefined length of time passing (e.g., a week, a month, a quarter, etc.), the training service 110 can retrain or tune the teacher model 135. In some embodiments, each time new training data is received, the teacher model 135 can be retrained. As the teacher model 135 is continuously re-trained, the knowledge learned by the teacher model 135 can periodically be distilled to the student model 140. In some embodiments, the teacher 135 is retrained at a shorter interval than the student model 140. For example, the teacher model 135 can be retrained on a weekly basis, while the knowledge of the teacher model 135 can be distilled to the student model 140 at a bi-weekly or monthly basis. In this regard, the student model 140 can be deployed (e.g., to the same or a different platform where the teacher model 135 is run). Because the teacher model 135 is not deployed, it can continuously train without interrupting the performance of the student model 140. Therefore, when information is periodically distilled to the student model 140, the student model 140 may be offline or unavailable for a shorter period of time than if the student model 140 had to train on the entire training dataset 115 that the training service 110 collects and uses to continuously retrain the teacher model 135. In some embodiments, the computing system 105 can compare predictions from a custom surgeon student model 140 with a standard model, e.g., a model trained with data of a large corpus of surgeons instead of being trained on data of one individual surgeon. The computing system 105 can compare surgeon predictions with another surgeon and map of deviations. The client device 173 can provide a graphical output illustrating deviations of predictions as a time series overlayed on video of medical procedure. The computing system 105 can use a timesformer architecture to determine the deviations.

Referring now to FIG. 2, among others, an example computing system 105 to train a machine learning model 120 using ontology knowledge and data of multiple modalities is shown. The model system 120 of FIG. 2 can be a multi-modal model that uses multiple data modalities of data of the robotic medical system 125 to classify segments of a medical procedure. The data modalities can include endoscopic video data 130, robotic kinematics data 205, event data of a robotic medical system, etc. Furthermore, the model system 120 of FIG. 2 can include multiple teacher models 255 (e.g., the teacher model 135 and the teacher model 210). For example, the model system 120 can include one teacher model to train and execute on data of each data modality. For example, the teacher model 135 can train and execute on video data 130, while the teacher model 210 can train and execute on robotic kinematics data 205. In FIG. 2, the model system 120 can provide collaborative learning of multiple teacher models 255 with multiple data modalities combined with surgical ontology knowledge.

The model 215 can be an embedding model or feature extraction model. The model 215 can be a timeseries transformer model, an encoder-decoder model, a recurrent neural network (RNN), a long-short term memory (LSTM) neural network, etc. The model 215 can include at least one timeseries classification model 215. The model 215 can receive the robotics kinematics data 205, and generate kinematics feature vectors 220 using the robotics kinematics 205. The kinematics data 205 can represent kinematics via multidimensional timeseries data where each dimension is a position or velocity vector of a certain robotic arm joint. The model 215 can generate a feature vector 220 for a window or time range of the kinematics data 205. In this regard, each feature vector 220 can correspond to a particular window or time range of the robotics kinematics data 205.

The teacher model 210 can include at least one model to predict gestures 225 from the feature vectors 220 produced from the robotics kinematics data 205. The teacher model 210 can include a support vector machine, a decision tree, a neural network, a convolutional neural network, a recurrent neural network, etc. to classify or predict the gestures 225 using the feature vectors 220. The teacher model 135 can directly make the phase predictions 155 from the video feature vectors 150, e.g., without using the mapping 230. While FIG. 2 depicts two teacher models 255, teacher model 135 and teacher model 210, the model system 120 can include any number of teacher models 255, each including a transformer, embedding, or feature extraction model to generate feature vectors for a different data modality. Furthermore, each teacher model can include a classification or prediction model that classifies a segment of the medical procedure using feature vectors produced by each respective transformer, embedding, or feature extraction model.

The teacher model 210 can classify the gesture classifications or predictions 225 from the kinematics feature vector 220 at a first level of a hierarchy in the ontology. The teacher model 210 can directly predict the gestures 225 from the kinematics feature vectors 220 without using the mapping 230. For example, in FIG. 2, the predictions 225 can be gesture predictions. The mapping 230 can map the predictions 225 from the first level of the hierarchy to a second level of the hierarchy. For example, in FIG. 2, the mapping 230 can be a gesture to phase mapping that translates the gesture predictions 225 to phase predictions 235. For example, the mapping 230 can indicate that a particular series of gestures 225 corresponds to one particular phase. The model system 120 can map, according to the ontology indicating the hierarchy represented in the mapping 230, segments classified in the first level of the hierarchy to segments of the second level of the hierarchy. The second level can be a higher level than the first level, e.g., the mapping can be gesture to action, or gesture to step, or gesture to phase, etc. The mapping 230 can produce converted predictions 235. In FIG. 2, the converted predictions can be phase predictions of the medical procedure.

The training service 110 can determine an ontology dependency loss 250. The model system 120 can determine an ontology dependency loss 250 using the converted prediction 235 and the ground truth 175. The model system 120 can use truth data of the training dataset 115 to generate a loss 250 using the mapped segment, and train the model 210 using the resulting loss 250. The loss 250 can be mean absolute error (MAE), mean squared error (MSE), cross-entropy loss, etc. The training service 110 can determine the loss 250 based on the converted predictions 235 and ground truth 175. For example, the training service 110 can compare the converted predictions 235 with the ground truth 175 to compute the loss. The ground truth 175 can be a predetermined classification of the segment. The ground truth 175 can be a classification of the segment in the same level of the hierarchy as the converted prediction 165. For example, the ground truth 175 can indicate the actual phase for the segment that the teacher model 135 converted the gesture predictions 225 to. For example, the converted prediction 235 can be a predicted phase for the segment, while the ground truth 175 can indicate the actual phase for the segment.

Each of the teacher models 255 can include a loss, such as a cross-entropy loss, to train the respective teacher model. For example, the teacher model 135 can include a phase loss 265 while the teacher model 210 can include a gesture loss 245. These losses can be determined from direct predictions of each model, e.g., a phase prediction 155 or a gesture prediction 225 that is determined without using the mapping 230. The training service 110 can determine the loss 245 using the predictions 225 and ground truth 240. The loss 245 can be mean absolute error (MAE), mean squared error (MSE), cross-entropy loss, etc. The training service 110 can compare the predictions 225 with the ground truth 240 to compute the loss 245. The ground truth 240 can be a predetermined classification of the segment. The ground truth 240 can be a classification of the segment in the same level of the hierarchy as the prediction 225. For example, the ground truth 240 can indicate an actual gesture for the segment that the teacher model 135 directly predicted. For example, the prediction 225 can be a predicted gesture for the segment, while the ground truth 240 can indicate the actual gesture for the segment.

The teacher model 135 can include the video transformer 145 to generate the video feature vectors 150 from the video 130. The teacher model 135 can include at least one model that makes a phase prediction 155 from the video feature vectors 150. The prediction 155 can be a prediction of the phase in the ontology that the feature vectors 150 correspond to. The training service 110 can determine a loss 265 using the predictions 155 and ground truth 175. The loss 265 can be mean absolute error (MAE), mean squared error (MSE), cross-entropy loss, etc. The training service 110 can compare the predictions 155 with the ground truth 175 to compute the loss 265. The ground truth 175 can be a predetermined classification of the segment. The ground truth 240 can be a classification of the segment in the same level of the hierarchy as the prediction 155. For example, the ground truth 175 can indicate an actual phase for the segment that the teacher model 135 directly predicted. For example, the prediction 155 can be a predicted phase for the segment, while the ground truth 175 can indicate the actual phase for the segment.

The training service 110 can train multiple teacher models 255 (e.g., the teacher model 135 and the teacher model 210) simultaneously, or individually to produce a shared knowledge of all surgical data. For example, when training the multi-modality teachers 255 separately, the training service 110 can train the teacher models 255 on each modality separately so that feature representations of each modality can be effectively extracted. Then, the representations from all modalities can be fused by the training service 110 in different ways by combining all the supplementary information. For example, the training service 110 can concatenation with the equal importance factor of different modalities, or a weighted linear combination to combine the supplementary information based on the relative importance of individual modalities.

The training service 110 can train the multi-model teacher models 255 together or simultaneously. The training service 110 can encourage teachers that specialize different data modalities to simultaneously adjust their parameters to achieve an overall learning objective, e.g., recognizing surgical phases, while leveraging the hierarchical relations of surgical ontology. Since different modalities are extracted from different domains of surgical data and feature extractors the representations of the modalities should be distinct from each other. To quantify this distinction, the model system 120 can include a multi-modal teacher similarity loss 260. The multi-modal similarity loss 260 can quantify the similarity of extracted features between teacher models 135 and 210 of different data modalities.

The loss 260 can be a distance measure between at least one feature vector 150 and at least one corresponding feature vector 220. The loss 260 can be Euclidean distance, Manhattan Distance, Cosine Similarity, Kullback-Leibler (KL) divergence, etc. The loss 260 can measure how similar or different the feature vectors produced by the teacher model 135 and the teacher model 210 are respectively. For example, the training service 110 can compare video feature vectors 150 against the kinematics feature vectors 220 to determine the loss 260. The training service 110 can compare video feature vectors 150 and the kinematics feature vectors 220 of the same time period, same window, or corresponding to the same point in time.

The training service 110 can penalize a similarity between the feature vectors extracted from distinct data sources to provide an expectation that the feature vectors produced from data of different modalities be distinct. For example, the training service 110 can operate to minimize or decrease the similarity loss 260 to make the feature vectors 150 and 220 less similar and more distinct. The training service 110 can increase or maximize a dissimilarity between the first feature vectors 150 and the kinematics feature vectors 220. This can cause the teacher models 255 to learn and use dissimilar data sources differently, while simultaneously making contributions to the shared knowledge between the teacher models 255. The training service 110 can train, adjust, or update the parameters or weights of the embedding or feature extraction models (e.g., the video transformer 145 or the timeseries classification model 215) to maximize or increase the differences between the feature vectors produced by the teacher model 135 and the teacher model 210 respectively.

The training service 110 can fully train the teacher model 135 and the teacher model 210 to have knowledge of different data modalities and surgical ontologies. Once the teacher models 255 are trained, the training service 110 can train and fine tune the student model 140 using all the teacher models 255. The training service 110 can train the student model 140 using the phase loss 193, and further train or fine-tune the video transformer 190 of the student model 140 using the teacher-student distillation loss 187.

The training service 110 can distill knowledge from a teacher model 135 to the student model 140 that is trained on the same data modality as the teacher model 135. For example, both the student model 140 and the teacher model 135 can be trained on a single common data modality, e.g., video data 130. The student model 140 can have knowledge distilled to the embedding or feature extraction model 190 from an embedding or feature extraction model 145 of a teacher model 135 trained on the same data modality as the embedding or feature extraction model 190.

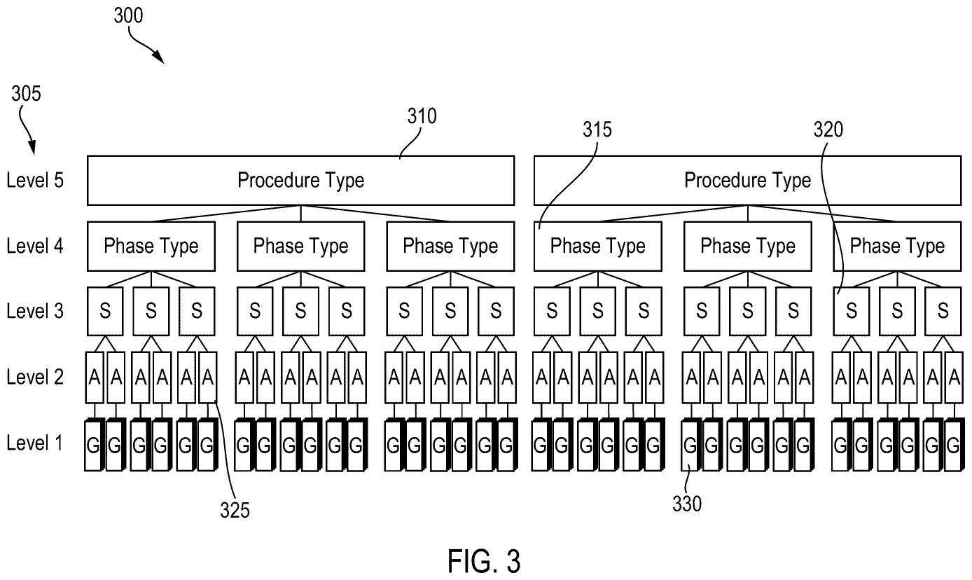

Referring now to FIG. 3, among others, an ontology 300 of different segment types of a medical procedure organized in a hierarchy is shown. The ontology 300 can define available segment types or classes for the model system 120 to classify a segment or portion of a medical procedure video 130 into. The student model 140, the teacher model 135, or the teacher model 210 can classify segments into the available segment types or available segment classes defined in the ontology 300. For example, the predictions 155, the predictions 225, or the predictions 197 can be predictions of segment types or classes defined in the ontology 300.

The ontology 300 can include a variety of levels 305 forming a hierarchy. In FIG. 3, the ontology 300 can include a procedure level, a phase level, a step level, an action level, and a gesture level. However, the ontology 300 can include any number of levels, e.g., the ontology 300 can include a phase level, a step level, and an action level or the ontology 300 can include an action level and a gesture level. The hierarchy of the ontology 300 can define which segment types make up other segment types. For example, for a given procedure type 310, the ontology 300 can identify what phase types 315 can be part of the procedure type 310. For example, if the procedure type 310 an appendectomy, the phase types 315 for the appendectomy could be incisions, appendix removal, closure, and sterilization.

The procedure type 310 can indicate the specific surgical or medical procedure, e.g., colonoscopy, appendicitis, hernia repair, breast biopsy, etc. The phase types 315 can be universal surgical phases that divide the surgical procedure into distinct phases that can be commonly found across different types of surgical procedures, such as exposure, dissection, transection, reconstruction, and extraction. The tasks or steps 320 can further breaks down each phase 315 into procedure-specific tasks, such as dissection of calots triangle, ligation/division of cystic duct, ligation/division of cystic artery in a cholecystectomy, etc. Actions 325 or atomic gestures 330 can be the smallest units of surgical activities, enabling the model system 120 to understand precise movements and gestures within each task or step 320. This might be further separated into two categories as well, e.g., actions 325 and gestures 330. For example, the actions 325 could be suturing, knot tying, etc., whereas the gesture 330 could be sweeping, grasping, or something even more atomic.

For a given phase type 315, the ontology 300 can identify what step types 320 can be part of the phase type 315. For example, if the procedure type 310 an appendectomy and the phase 315 is appendix removal, the step types 320 could be locating an appendix, tying the appendix off, and removing the appendix. For a given step type 320, the ontology 300 can identify what actions types 325 can be part of the step type 320. For example, if the procedure type 310 is an appendicitis, the phase 315 is appendix removal, and the step 320 is removing the appendix, the action types 325 can be separating the appendix from the intestine, placing the appendix in a specimen bag within the patient, removing the bagged appendix from the patient, etc. Furthermore, each action type 325 can have various gestures. The gestures can be individual movements of surgical instruments, robotic arms, or endoscopes to complete each respective action 325.

Mappings of the model system 120, e.g., the mapping 160 or the mapping 230, can represent or be based on the ontology 300. For example, the mappings 160 or 230 can provide translations, transformations, relationships, or mappings between the various levels 305. For example, the mappings 160 or 230 can be a matrix that translates between one, two, three, or more levels 305 of the hierarchy. The ontology 300 can be predefined or preprogrammed. For example, a user can provide input via the client device 173 defining or specifying the levels 305 of the ontology, and what procedures, phases, steps, actions, or gestures are available for each level. The computing system 105 can generate the mapping 160 or 230 from the ontology 300. In some embodiments, the user can provide the mapping 160 or 230 directly via the client device 173.

Referring generally to FIGS. 1-3, the segment classifications can be used by the computing system 105 (or the medical robotic system 125 or the client device 173) to generate objective performance indicators (OPIs) and/or practitioner fingerprints or signatures. The computing system 105 can generate at least one OPI. The OPI can represent performance, operation, or quality of at least one of the surgeon, a surgical team, a robotic surgical system, a surgical session, etc. The OPI can represent the performance of specific surgeons, a specific medical robot, the patient outcome for a particular surgery, etc. A surgery can be formed from phases, which can be made up of steps, which can be made up of actions. The OPIs can be generated for specific surgeries, specific phases, specific steps, or specific actions. The OPI can be a binary value, a value within a range, a percentage or any other numeric value. The OPI can be a raw value, or a normalized value. The OPI can be normalized for different surgeons, hospitals, surgery types, medical equipment, specific time ranges (days, months, years), etc. The OPI can be a metric, statistic, count, value, indicator, color, grade, vector, or function. The OPI can be produced from raw data, or from a combination of other OPIs. The OPIs generated by the computing system 105 can include, but not limited to, at least one of energy usage, pedal count, tool clutch count, surgical duration, total instrument path length, total instrument angular path length, or hand controller clutch count.

For example, the computing system 105 can receive the classifications of various procedure types, phases, actions, or gestures, and determine various OPIs from the segment classifications. For example, the classifications can indicate starting times, ending times, or lengths of the various classified segments. The computing system 105 can store lengths of times for various segments, and compare the classified segments against the stored benchmark or nominal lengths of time to generate a score or indicator that indicates how well the medical practitioner performed. For example, if a particular phase of a particular type of medical procedure typically takes 25 minutes, but the surgeon completed said phase in 35 minutes, a score value can be generated that indicates that the surgeon was inefficient, or indicates that the surgeon is not as experienced or skilled as the benchmark surgeon.

Furthermore, the segment classifications can count the number of gestures. For example, a particular action may need a particular number and type of gestures, and additional gestures may result in inefficiencies, worse patient outcomes, etc. For example, it may take nominally take two gestures to make an incision. If the surgeon makes two or three gestures to make the incision, this can indicate good surgeon performance, but if a practitioner takes 10 gestures to make the same incision, this may indicate poor surgeon performance. The computing system 105 can compare the count and type of gestures against benchmark or nominal counts and types of gestures to determine OPIs.

Furthermore, the computing system 105 can analyze the patterns of gestures, actions, or steps. For example, if a surgeon has to repeat steps, this may indicate that the first step was not performed correctly. For example, if a surgeon cauterizes a wound, but then later in the procedure cauterizes the same wound again, this may indicate that the surgeon did not cauterize the wound correctly on the first attempt. Various performance scores or OPIs can be generated to take into account the pattern of gestures, actions, steps, or phases of the medical procedure.

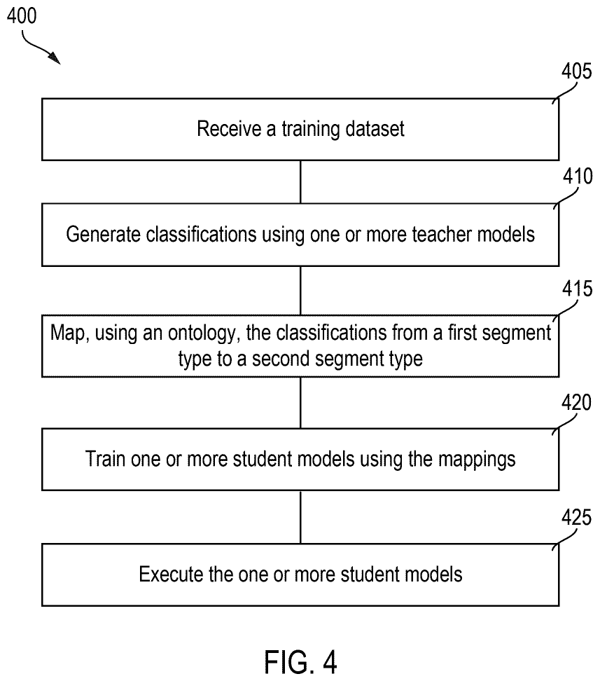

Referring now to FIG. 4, among others, an example method 400 of training a machine learning model using ontology knowledge is shown. The system 100, the computing system 105, the medical robotic system 125, the client device 173, the training service 110, or the inference service 183 can perform at least a portion of the method 400. The method 400 can include an ACT 405 of receiving a training dataset. The method 400 can include an ACT 410 of generating classifications using one or more teacher models. The method 400 can include an ACT 415 of mapping, using an ontology, the classifications from a first segment type to a second segment type. The method 400 can include an ACT 420 of training one or more student models using the mappings. The method 400 can include an ACT 425 of executing the one or more student models.

At ACT 405, the method 400 can include receiving, by the computing system 105, a training dataset 115. The method 400 can include receiving the training dataset 115 from the medical robotic system 125. The method 400 can include receiving the training dataset 115 from the client device 173. The method 400 can include storing the training dataset 115 for training the model system 120. The training service 110 can periodically update the training dataset 115 as new data is received. For example, as new training data is received (e.g., new samples and classifications for the samples) from the medical robotic systems 125 or the client device 173, the training service 110 can update the training dataset 115 for periodic or continuous training, re-training, or tuning of the model system 120. The method 400 can include receiving a training dataset 115 including a single data modality, or data of a variety of data modalities, e.g., video data 130, kinematics data 205, event data, OR data, etc. Furthermore, the training dataset 115 can include truth data, e.g., classifications or labels for various segments of the medical procedure. The labels for the various segments can be labels in the ontology 300, e.g., procedure types 310, phase types 315, step types 320, action types 325, and/or gesture types 330.

At ACT 410, the method 400 can include generating, by the computing system 105, classifications using one or more teacher models. The training service 110 can apply samples to the teacher model 135 and/or the teacher model 210 to generate a classification or prediction for the sample. Each sample can be a different segment or window of the medical procedure or the video 130. Each sample can include data of a single data modality, or multiple data modalities, e.g., video data 130, the kinematics data 205, event data, OR data, etc.

The method 400 can include executing an embedding model, feature extraction model, or transformer of each of the one or more teacher models to generate feature vectors. For example, the teacher model 135 can execute the video transformer 145 with the video 130 as an input to produce the feature vectors 150. Similarly, the teacher model 210 can execute the timeseries classification model 215 to generate the kinematics feature vectors 220. Furthermore, the method 400 can include executing a prediction or classification model of each teacher model using the generated feature vectors to generate a classification for the segment of the medical procedure. For example, the teacher model 135 can generate a step prediction 155 from the feature vectors 150. The teacher model 210 can generate a gesture prediction 225 from the kinematics feature vector 220.

At ACT 415, the method 400 can include mapping, by the computing system 105, using an ontology, the classifications from a first segment type to a second segment type. The first and second segment types can be the types defined in the ontology 300 at different levels 305. For example, the first segment type could be the gesture types 330 while the second segment type could be the phase segment types 315. The classifications generated at ACT 410 can be of a first segment type in a first level. For example, the teacher model 135 can generate step predictions 155, which can be predictions in a third level in the hierarchy of the ontology 300. The teacher model 135 can include a mapping 160. The mapping 160 can map, transform, translate, or convert the step predictions 155 into the phase predictions 165. The phase predictions 165 can be segment types in a fourth level of the hierarchy of the ontology 300. The mapping 160 can convert the step predictions 155 to different phase predictions 165. For example, one step prediction 155 can be mapped to a first phase type, while a second step prediction can be mapped to a second phase type. For example, a step of suturing can be mapped to a phase of closing and cleaning a patient, while a step of creating an incision can be mapped to a phase of opening a patient and beginning a procedure.

Furthermore, the teacher model 210 can generate gesture predictions 225, which can be in a first or lowest level of the hierarchy of the ontology 300. The teacher model 210 can include a mapping 230 that converts the gesture predictions 225 to phase predictions 235. For example, the mapping 230 can convert gesture predictions 225 which can be gesture types 330 from the first level to the fourth level of gesture predictions 225 which can be phase types 315.

At ACT 420, the method 400 can include training, by the computing system 105, one or more student models 140 using the mappings. The mappings can be the mappings generated at ACT 415. The mappings can be the converted predictions, e.g., the phase predictions 235 or the phase predictions 165. With these mappings, the method 400 can include determining or calculating a loss 170 or 250 using the mapping. For example, the training service 110 can compare the converted phase predictions 165 with phase ground truth data 175. The training service 110 can compare the converted phase predictions 165 with the actual phases of the corresponding segments of the medical procedure indicated by the ground truth 175. The training service 110 can train the video transformer 145 (or the prediction model of the teacher 135, using the loss 170. Furthermore, the knowledge of the video transformer 145 can be distilled to the video transformer 190 of the student model 140 using a distillation loss 187. In this regard, the student model 140 can be trained from the mappings, e.g., indirectly through the distillation loss 187. In some embodiments, the distillation via the distillation loss 187 can be performed after the teacher model 135 or 210 is finished or concluded. In some embodiments, the student model 140 is trained and knowledge is distilled for the student model 140 while the teacher model 135 or 210 are being trained.

At ACT 425, the method 400 can include executing, by the computing system 105, the one or more student models 140. The method 400 can include training the student model 140 as part of the overall training of the teacher model 135 or the teacher model 210. The method 400 can include training the student model 140 separately from the teacher model 135 or the teacher model 210. Once the student model 140 is fully trained (e.g., a predefined number of training epochs have been completed, a predefined length of time has passed, the student model 140 reaches a predefined accuracy level, etc.) the training service 110 can cause the student model 140 to be deployed. The inference service 183 can cause the student model 140 to be executed locally on the computing system 105, or alternatively execute directly on the medical robotic system 125 or the client device 173.

The method 400 can include receiving or collecting inference data from the medical robotic system 125, e.g., actual cases or samples for the model system 120 to break into segments and classify. The inference data can include endoscope videos, kinematics data, event data, OR data, etc. The inference service 183 can cause the deployed student model 140 to execute on the collected information to generate classifications or labels for various segments of the medical procedure. In some embodiments, the model system 120 can include one or multiple different student models 140, e.g., one student model 140 to classify segments of the medical procedure into a segment type of a level 305 of the hierarchy of the ontology 300. The student models 140 can execute to produce the classifications. The inference service 183 can use the output 177 of the student models 140 to generate a video 130 of the medical procedure that includes one or multiple timelines that identify the current gesture, action, step, phase, or procedure type for a given point of time in the video. For example, the inference service 183 can generate at least one timeseries. The timeseries can indicate timestamps indicating the different gesture types 330, action types 325, step types 320, and procedure types 310. The procedure type 310 can be a flag or label for the entire video 130, and may not be a timeseries. The resulting labeled video 130 can be viewed or reviewed on the client device 173.

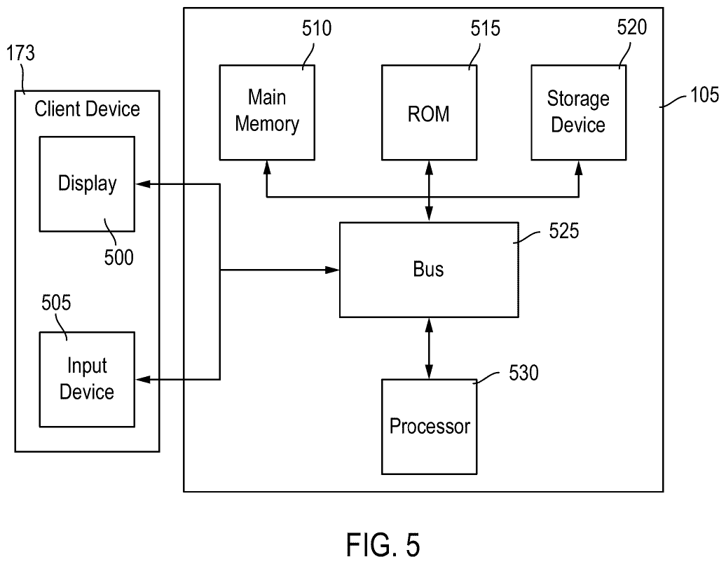

Referring now to FIG. 5, among others, an example block diagram of a computing system 105 is shown. The computing system 105 can include or be used to implement a data processing system or its components. The architecture described in FIG. 5 can be used to implement the computing system 105, the medical robotic system 125, or the client device 173. The computing system 105 can include at least one bus 525 or other communication component for communicating information and at least one processor 530 or processing circuit coupled to the bus 525 for processing information. The computing system 105 can include one or more processors 530 or processing circuits coupled to the bus 525 for processing information. The computing system 105 can include at least one main memory 510, such as a random access memory (RAM) or other dynamic storage device, coupled to the bus 525 for storing information, and instructions to be executed by the processor 530. The main memory 510 can be used for storing information during execution of instructions by the processor 530. The computing system 105 can further include at least one read only memory (ROM) 515 or other static storage device coupled to the bus 525 for storing static information and instructions for the processor 530. A storage device 520, such as a solid state device, magnetic disk or optical disk, can be coupled to the bus 525 to persistently store information and instructions.

The computing system 105 can be coupled via the bus 525 to a display 500, such as a liquid crystal display, or active matrix display. The display 500 can display information to a user. An input device 505, such as a keyboard or voice interface can be coupled to the bus 525 for communicating information and commands to the processor 530. The input device 505 can include a touch screen of the display 500. The input device 505 can include a cursor control, such as a mouse, a trackball, or cursor direction keys, for communicating direction information and command selections to the processor 530 and for controlling cursor movement on the display 500. The display 500 and the input device 505 can be a component of the client device 173 coupled with the computing system 105.

The processes, systems and methods described herein can be implemented by the computing system 105 in response to the processor 530 executing an arrangement of instructions contained in main memory 510. Such instructions can be read into main memory 510 from another computer-readable medium, such as the storage device 520. Execution of the arrangement of instructions contained in main memory 510 causes the computing system 105 to perform the illustrative processes described herein. One or more processors in a multi-processing arrangement can be employed to execute the instructions contained in main memory 510. Hard-wired circuitry can be used in place of or in combination with software instructions together with the systems and methods described herein. Systems and methods described herein are not limited to any specific combination of hardware circuitry and software.

Although an example computing system has been described in FIG. 5, the subject matter including the operations described in this specification can be implemented in other types of digital electronic circuitry, or in computer software, firmware, or hardware, including the structures disclosed in this specification and their structural equivalents, or in combinations of one or more of them.

Some of the description herein emphasizes the structural independence of the aspects of the system components or groupings of operations and responsibilities of these system components. Other groupings that execute similar overall operations are within the scope of the present application. Modules can be implemented in hardware or as computer instructions on a non-transient computer readable storage medium, and modules can be distributed across various hardware or computer based components.

The systems described above can provide multiple ones of any or each of those components and these components can be provided on either a standalone system or on multiple instantiations in a distributed system. In addition, the systems and methods described above can be provided as one or more computer-readable programs or executable instructions embodied on or in one or more articles of manufacture. The article of manufacture can be cloud storage, a hard disk, a CD-ROM, a flash memory card, a PROM, a RAM, a ROM, or a magnetic tape. In general, the computer-readable programs can be implemented in any programming language, such as LISP, PERL, C, C++, C #, PROLOG, Python, or in any byte code language such as JAVA. The software programs or executable instructions can be stored on or in one or more articles of manufacture as object code.

Example and non-limiting module implementation elements include sensors providing any value determined herein, sensors providing any value that is a precursor to a value determined herein, datalink or network hardware including communication chips, oscillating crystals, communication links, cables, twisted pair wiring, coaxial wiring, shielded wiring, transmitters, receivers, or transceivers, logic circuits, hard-wired logic circuits, reconfigurable logic circuits in a particular non-transient state configured according to the module specification, any actuator including at least an electrical, hydraulic, or pneumatic actuator, a solenoid, an op-amp, analog control elements (springs, filters, integrators, adders, dividers, gain elements), or digital control elements.

The subject matter and the operations described in this specification can be implemented in digital electronic circuitry, or in computer software, firmware, or hardware, including the structures disclosed in this specification and their structural equivalents, or in combinations of one or more of them. The subject matter described in this specification can be implemented as one or more computer programs, e.g., one or more circuits of computer program instructions, encoded on one or more computer storage media for execution by, or to control the operation of, data processing apparatuses. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. While a computer storage medium is not a propagated signal, a computer storage medium can be a source or destination of computer program instructions encoded in an artificially generated propagated signal. The computer storage medium can also be, or be included in, one or more separate components or media (e.g., multiple CDs, disks, or other storage devices including cloud storage). The operations described in this specification can be implemented as operations performed by a data processing apparatus on data stored on one or more computer-readable storage devices or received from other sources.

The terms “computing device”, “component” or “data processing apparatus” or the like encompass various apparatuses, devices, and machines for processing data, including by way of example a programmable processor, a computer, a system on a chip, or multiple ones, or combinations of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, app, script, or code) can be written in any form of programming language, including compiled or interpreted languages, declarative or procedural languages, and can be deployed in any form, including as a stand-alone program or as a module, component, subroutine, object, or other unit suitable for use in a computing environment. A computer program can correspond to a file in a file system. A computer program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatuses can also be implemented as, special purpose logic circuitry, e.g., an FPGA (field programmable gate array) or an ASIC (application specific integrated circuit). Devices suitable for storing computer program instructions and data can include non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

The subject matter described herein can be implemented in a computing system that includes a back end component, e.g., as a data server, or that includes a middleware component, e.g., an application server, or that includes a front end component, e.g., a client computer having a graphical user interface or a web browser through which a user can interact with an implementation of the subject matter described in this specification, or a combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While operations are depicted in the drawings in a particular order, such operations are not required to be performed in the particular order shown or in sequential order, and all illustrated operations are not required to be performed. Actions described herein can be performed in a different order.

Having now described some illustrative implementations, it is apparent that the foregoing is illustrative and not limiting, having been presented by way of example. In particular, although many of the examples presented herein involve specific combinations of method acts or system elements, those acts and those elements may be combined in other ways to accomplish the same objectives. ACTs, elements and features discussed in connection with one implementation are not intended to be excluded from a similar role in other implementations.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any ACT or element being based on any information, act or element may include implementations where the act or element is based at least in part on any information, act, or element.

Any implementation disclosed herein may be combined with any other implementation or example, and references to “an implementation,” “some implementations,” “one implementation” or the like are not necessarily mutually exclusive and are intended to indicate that a particular feature, structure, or characteristic described in connection with the implementation may be included in at least one implementation or example. Such terms as used herein are not necessarily all referring to the same implementation. Any implementation may be combined with any other implementation, inclusively or exclusively, in any manner consistent with the aspects and implementations disclosed herein.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system, comprising:
one or more processors, coupled with memory, to:
receive a training dataset related to medical procedures performed by one or more robotic medical systems by a plurality of medical practitioners;
generate, using the training dataset and one or more teacher models, classifications of segments of the medical procedures in a first segment type;
map, using an ontology indicating a hierarchy of different segment types of the medical procedures, the classifications of the segments in the first segment type to a second segment type;
train, using the mapping based on the classifications generated by the one or more teacher models and data associated with a single medical practitioner, one or more student models with a machine learning technique;
execute, using data received from a robotic medical system for a medical procedure, the one or more student models to generate a classification of a segment of the medical procedure performed by the single medical practitioner; and
cause a graphical user interface to display the classification of the segment.

2. The system of claim 1, wherein the ontology indicates a plurality of levels of the different segment types, wherein the plurality of levels include at least two of:
a first level of a plurality of actions and a mapping indicating a plurality of steps that the plurality of actions map to;
a second level of the plurality of steps and a mapping indicating a plurality of phases that the plurality of steps map to; and
a third level of the plurality of phases.

3. The system of claim 1, wherein the one or more teacher models store the ontology as at least one matrix.

4. The system of claim 1, wherein the one or more processors are further configured to:
train a teacher model of the one or more teacher models to classify the segments of the medical procedure;
distill the training of the teacher model to a student model of the one or more student models; and
execute, using the data received from the one or more robotic medical systems for the medical procedure, the student model to classify the segment of the medical procedure.

5. The system of claim 4, wherein the one or more processors are further configured to:
train the teacher model using first data of the training dataset describing first medical procedures performed by the plurality of medical practitioners via the one or more robotic medical systems;
distill the training of the teacher model using the first data to the student model;
train the student model using second data of the training dataset describing second medical procedures performed by the single medical practitioner via the one or more robotic medical systems; and
execute, using the data received from the one or more robotic medical systems for the medical procedure performed by the single medical practitioner using the one or more robotic medical systems, the student model to classify the segment of the medical procedure.

6. The system of claim 4, wherein the one or more processors are further configured to:
determine, using a first teacher model of the one or more teacher models and the training dataset of a first data modality, a plurality of first features;
generate, using the first teacher model, first teacher classifications of the segments using the plurality of first features;
determine, using a second teacher model of the one or more teacher models and the training dataset of a second data modality, a plurality of second features;
generate, using the second teacher model, second teacher classifications of the segments using the plurality of second features; and
train the first teacher model and the second teacher model using one or more losses determined from the first teacher classifications of the first teacher model and the second teacher classifications of the second teacher model.

7. The system of claim 6, wherein the one or more processors are further configured to:
classify, using the first teacher model, the segments directly from the plurality of first features;
determine a first loss from the classified segments of the first teacher model and the training dataset;
train the first teacher model using the first loss.

8. The system of claim 7, wherein the one or more processors are further configured to:
classify, using the second teacher model, the segments of a first level of the hierarchy from the plurality of second features;
map, according to the ontology indicating the hierarchy, the segments of the first level of the hierarchy to segments of a second level of the hierarchy, wherein the second level is higher than the first level in the hierarchy;
determine a second loss from the classified segments of the second teacher model and the training dataset; and
train the second teacher model using the second loss.

9. The system of claim 6, wherein the one or more processors are further configured to:
compare the plurality of first features with the plurality of second features; and
train the first teacher model and the second teacher model to increase a dissimilarity between the plurality of first features and the plurality of second features.

10. The system of claim 1, wherein the one or more processors are further configured to:
classify, using a teacher model of the one or more teacher models, segments of a first level of the hierarchy;
map, according to the ontology indicating the hierarchy, the classified segments of the first level of the hierarchy to segment types of a second level of the hierarchy, wherein the second level is higher than the first level in the hierarchy;
determine a loss using the classified segments mapped to the segment types of the second level of the hierarchy and the training dataset; and
train the teacher model using the loss.

11. The system of claim 10, wherein:
the segments of the first level of the hierarchy are steps of phases of the medical procedures; and
the segments of the second level of the hierarchy are the phases of the medical procedures.

12. The system of claim 10, wherein the teacher model of the one or more teacher models includes:
a first embedding model to generate a plurality of first feature vectors from the training dataset; and
a first model to classify the segments of the first level from the plurality of first feature vectors;
wherein a student model of the one or more student models includes:
a second embedding model to generate a plurality of second feature vectors from the training dataset; and
a second model to classify the segments of the first level from the plurality of second feature vectors;
wherein the one or more processors are configured to:
determine a first loss for the teacher model based on the segments classified by the first model and the training dataset;
train the teacher model using the first loss;
determine a second loss for the student model based on the segments classified by the second model and the training dataset; and
train the student model using the second loss.

13. The system of claim 12, wherein the one or more processors are further configured to:
compare the plurality of first feature vectors with the plurality of second feature vectors to generate a third loss; and
distill training of the teacher model to the student model using the third loss.

14. The system of claim 13, wherein the one or more processors are further configured to:
generate a plurality of distance measures between the plurality of first feature vectors and the plurality of second feature vectors; and
update at least one parameter of the second embedding model of the student model to decrease the plurality of distance measures.

15. A method, comprising:
receiving, by one or more processors, coupled with memory, a training dataset describing medical procedures performed by one or more robotic medical systems by a plurality of medical practitioners;
training, by the one or more processors, using the training dataset and an ontology indicating a hierarchy of different segment types of the medical procedures, a teacher model to classify segments of the medical procedures;
distilling, by the one or more processors, the training of the teacher model to a student model trained to classify the segments of the medical procedures for a single medical practitioner;
executing, using data received from a one or more robotic medical system for a medical procedure, the student model to generate a classification of a segment of the medical procedure for the single medical practitioner; and
causing, by the one or more processors, a graphical user interface to display the classification of the segment of the medical procedure.

16. The method of claim 15, comprising:
determining, by the one or more processors, using a first teacher model and the training dataset of a first data modality, a plurality of first features;
generating, by the one or more processors, using the first teacher model, first teacher classifications of the segments using the plurality of first features;
determining, by the one or more processors, using a second teacher model and the training dataset of a second data modality, a plurality of second features;
generating, by the one or more processors, using the second teacher model, second teacher classifications of the segments using the plurality of second features; and
training, by the one or more processors, the first teacher model and the second teacher model using one or more losses determined from the first teacher classifications of the first teacher model and the second teacher classifications of the second teacher model.

17. The method of claim 16, wherein the first data modality or the second data modality are a video data modality, a kinematics data modality, an event data modality.

18. The method of claim 16, comprising:
classifying, by the one or more processors, using the first teacher model, the segments directly from the plurality of first features;
determining, by the one or more processors, a first loss from the classified segments of the first teacher model and the training dataset;
training, by the one or more processors, the first teacher model using the first loss;
classifying, by the one or more processors, using the second teacher model, the segments of a first level of the hierarchy from the plurality of second features;
mapping, by the one or more processors, according to the ontology indicating the hierarchy, the classified segments of the first level of the hierarchy to segment types of a second level of the hierarchy, wherein the second level is higher than the first level in the hierarchy;
determining, by the one or more processors, a second loss from the classified segments mapped to the segment types of the second level and the training dataset; and
training, by the one or more processors, the second teacher model using the second loss.

19. A non-transitory computer-readable medium storing processor-executable instructions that, when executed by one or more processors, cause the one or more processors to:
receive a training dataset related to medical procedures performed by one or more robotic medical systems by a plurality of medical practitioners;
generate, using the training dataset and one or more teacher models, classifications of segments of the medical procedures in a first segment type;
map, using an ontology indicating a hierarchy of different segment types of the medical procedures, the classifications of the segments in the first segment type to a second segment type;
train, using the mapping based on the classifications generated by the one or more teacher models and data associated with a single medical practitioner, one or more student models with a machine learning technique;
execute, using data received from a robotic medical system for a medical procedure, the one or more student models to generate a classification of a segment of the medical procedure performed by the single medical practitioner; and
cause a graphical user interface to display the classification of the segment.

20. The non-transitory computer-readable medium of claim 19, wherein the ontology indicates a plurality of levels of different segment types, wherein the plurality of levels include at least two of:
a first level of a plurality of actions and a mapping indicating a plurality of steps that the plurality of actions map to;
a second level of the plurality of steps and a mapping indicating a plurality of phases that the plurality of steps map to; and
a third level of the plurality of phases.
